(19) United States
(12) Patent Application Publication
(10) Pub. No.: US 2017/0001001 A1
(43) Pub. Date: Jan. 5, 2017

(54) LEFT SIDE SINGLE PASS LEAD FOR LA AND LV SENSING AND PACING

(71) Applicant: Cardiac Pacemakers, Inc., St. Paul, MN (US)

(72) Inventors: Lili Liu, Maple Grove, MN (US); Ronald W. Kunkel, Jim Falls, WI (US); Kyle K. Hoecke, Lino Lakes, MN (US); Kimberly A. Miklos, Minneapolis, MN (US); Matthew J. Miller, Stillwater, MN (US)

(21) Appl. No.: 15/199,855

(22) Filed: Jun. 30, 2016

Related U.S. Application Data
(60) Provisional application No. 62/187,800, filed on Jul. 1, 2015.

(51) Int. Cl. A61N 1/05; A61N 1/365; A61N 1/368; A61N 1/375; A61N 1/362

(52) U.S. Cl. CPC A61N 1/059; A61N 1/057; A61N 1/3752; A61N 1/3627; A61N 1/3684; A61N 1/36514; A61N 2001/0585

(57) ABSTRACT

Various aspects of the present disclosure are directed toward apparatuses, systems, and methods that include a single pass implantable lead configured to be coupled to the implantable medical device and arranged on a left ventricle and left atrium of a patient. The lead may include a proximal region having at least one electrode, a distal region having at least one electrode, and an intermediate region therebetween. The system can sense and pace the left atrium and the left ventricle.

### CROSS-REFERENCE TO RELATED APPLICATION

This application claims priority to Provisional Application No. 62/187,800 filed Jul. 1, 2015, which is herein incorporated by reference in its entirety.

### TECHNICAL FIELD

The present invention relates to medical devices and methods for stimulating/sensing a patient's heart. More specifically, the invention relates to devices and methods for stimulating/sensing the left atrium and left ventricle of the heart in a minimally-invasive manner.

### BACKGROUND

Implantable medical devices, such as electrical stimulators or sensors, are used in a variety of therapeutic applications. In some implantable medical devices, an electrical stimulator or sensor delivers electrical pulses to a target tissue site within a patient with the aid of one or more medical leads. The medical leads are coupled to the implantable medical device at one end while the other end carrying electrodes is placed at the target tissue site. The electrodes may be used in stimulating and/or sensing applications.

### SUMMARY

In Example 1, an apparatus comprising: an implantable medical device; and a single pass implantable lead configured to be coupled to the implantable medical device and arranged near or in a left ventricle and left atrium of a patient, the implantable medical lead comprising: a flexible lead body, a proximal region, a distal region, and an intermediate region between the proximal region and distal region; at least one proximal electrode arranged in the proximal region of the flexible lead body and configured to sense and pace the left atrium of the patient; and at least one distal electrode arranged in the distal region of the flexible lead body and configured to sense and pace the left ventricle of the patient.

In Example 2, the apparatus of Example 1, wherein the implantable medical device including a header having a connector port, and the single pass implantable lead is coupled to the implantable medical device via the connector port.

In Example 3, the apparatus of Example 2, wherein the implantable medical device is at least one of a Cardiac Resynchronization Therapy (CRT) device and a bradycardia pacemaker.

In Example 4, the apparatus of Examples 1-3, wherein the at least one proximal electrode is configured to sense and pace the left atrium of the patient in the at least one of great cardiac vein (GCV) and coronary sinus.

In Example 5, the apparatus of Examples 1-4, wherein at least one distal electrode is configured to sense and pace the left ventricle of the patient in the patient's coronary branch.

In Example 6, the apparatus of Examples 1-5, wherein at least one of the proximal region and the distal region having a pre-biased shape configured to engage a wall of a blood vessel to secure the lead.

In Example 7, the apparatus of Example 6, wherein the proximal region and the distal region comprise a pre-biased shape configured to engage a wall of a blood vessel to secure the lead.

In Example 8, the apparatus of Examples 1-7, wherein the intermediate region of the flexible lead body comprises an adjustable length, the adjustable length being configured to compress and stretch.

In Example 9, the apparatus of Example 8, wherein the adjustable intermediate region is configured to compress and stretch to mitigate displacement forces to enhance anchoring of the proximal region or distal region.

In Example 10, the apparatus of Example 8, wherein the adjustable intermediate region comprises a material composition having a lower durometer than a material composition of the proximal region and the distal region.

In Example 11, the apparatus of Examples 1-10, wherein the intermediate region is free of electrodes.

In Example 12, the apparatus of Examples 1-11, wherein the intermediate region comprises a first thickness, and the proximal and distal regions comprise a second thickness, and the first thickness differs from the second thickness.

In Example 13, the apparatus of Examples 1-12, wherein the intermediate length is between 40 mm and 150 mm.

In Example 14, the apparatus of Examples 1-13, wherein at least one of the proximal region, the distal region, and the intermediate region comprises polyurethane, and wherein at least one of the proximal region, the distal region, and the intermediate region comprises silicone.

In Example 15, the apparatus of Example 14, further comprising a material transition portion between the polyurethane and silicone, and wherein the material transition region is configured to provide a bias.

In Example 16, an apparatus comprising: an implantable medical device; and a single pass implantable lead configured to be coupled to the implantable medical device and arranged near or in a left ventricle and left atrium of a patient, the implantable medical lead comprising a flexible lead body having a proximal region configured to anchor the flexible lead body against an epicardial surface of a left atrial epicardial wall, a distal region configured to anchor the flexible lead body to a left ventricular epicardial wall, and an adjustable intermediate region between the proximal region and distal region; and at least one distal electrode arranged in the distal region of the flexible lead body and configured to sense and pace the left ventricle of the patient.

In Example 17, the apparatus of Example 16, wherein the implantable medical device including a header having a connector port, and the single pass implantable lead is coupled to the implantable medical device via the connector port.

In Example 18, the apparatus of Example 17, wherein the implantable medical device is at least one of a Cardiac Resynchronization Therapy (CRT) device and a bradycardia pacemaker.

In Example 19, the apparatus of Example 16, wherein the at least one proximal electrode is configured to sense and pace the left atrium of the patient in the GCV or coronary sinus, and wherein at least one distal electrode is configured to sense and pace the left ventricle of the patient in the patient's coronary branch.

In Example 20, the apparatus of Example 16, wherein the intermediate region of the flexible lead body comprises an adjustable length, the adjustable length being configured to compress and stretch.

In Example 21, the apparatus of Example 16, wherein the intermediate region comprises a first thickness, and the proximal and distal regions comprise a second thickness, and the first thickness differs from the second thickness.

In Example 22, the apparatus of Example 16, wherein the intermediate length is between 40 mm and 150 mm.

In Example 23, the apparatus of Example 16, wherein at least one of the proximal region, the distal region, and the intermediate region comprises polyurethane, and wherein at least one of the proximal region, the distal region, and the intermediate region comprises silicone.

In Example 24, the apparatus of Example 23, further comprising a material transition portion between the polyurethane and silicone, and wherein the material transition region is configured to provide a bias.

In Example 25, the apparatus of Example 24, wherein the material transition region is proximal to the at least one proximal electrode or the at least one distal electrode.

In Example 26, the apparatus of Example 25, wherein the material transition region is proximal to the at least one proximal electrode, and the at least one proximal electrode contacts the left atrium of the patient in the GCV or coronary sinus.

In Example 27, a single pass implantable lead comprising: a flexible lead body a proximal region configured to anchor the flexible lead body, a distal region configured to anchor the flexible lead body, and an adjustable intermediate region between the proximal region and distal region, the adjustable intermediate region being configured to mitigate displacement forces to enhance anchoring of the proximal region or distal region; at least one proximal electrode arranged in the proximal region of the flexible lead body and configured to sense and pace the left atrium of the patient; and at least one distal electrode arranged in the distal region of the flexible lead body and configured to sense and pace the left ventricle of the patient.

In Example 28, the single pass implantable lead of Example 27, wherein the adjustable intermediate region of the flexible lead body comprises an adjustable length.

In Example 29, the single pass implantable lead of Example 27, wherein the adjustable intermediate region is configured to compress and stretch to mitigate displacement forces to enhance anchoring of the proximal region or distal region.

In Example 30, the single pass implantable lead of Example 27, wherein at least one of the proximal region and the distal region having a pre-biased shape configured to engage a wall of a blood vessel to secure the lead.

In Example 31, the single pass implantable lead of Example 27, wherein the proximal region and the distal region comprise a pre-biased shape configured to engage a wall of a blood vessel to secure the lead.

In Example 32, a method of implanting a single pass implantable lead in a coronary venous system adjacent to a left ventricle and left atrium of a patient, the method comprising: arranging a proximal region the single pass implantable lead including at least one proximal electrode against the left atrium epicardial wall of the patient; arranging a distal region the single pass implantable lead including at least one distal electrode against the left ventricle epicardial wall of the patient, the single pass implantable lead having an adjustable intermediate region between the proximal region and the distal region; sensing or pacing a cardiac rhythm of the left atrium of the patient via the at least one proximal electrode; and sensing or pacing the left ventricle of the patient via the at least one distal electrode.

In Example 33, the method of Example 32, wherein arranging the proximal region comprises arranging the at least one proximal electrode in the patient's GCV or coronary sinus.

In Example 34, the method of Example 33, wherein arranging the distal region comprises arranging at least one distal electrode in the patient's coronary branch.

In Example 35, the method of Example 32, wherein further comprising coupling the single pass implantable lead to an implantable medical device, wherein the implantable medical device is at least one of a Cardiac Resynchronization Therapy (CRT) device and a bradycardia pacemaker.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic view of an implantable system including an exemplary implantable medical device and an implantable lead in accordance with embodiments of the present disclosure.

FIG. 2 is a schematic view of an exemplary single pass implantable lead in accordance with embodiments of the present disclosure.

FIG. 3A is an illustration of an exemplary placement of a single pass implantable lead in a patient's heart in accordance with embodiments of the present disclosure.

FIG. 3B is an illustration of another exemplary placement of a single pass implantable lead in a patient's heart in accordance with embodiments of the present disclosure.

FIG. 4A is a schematic view of an exemplary single pass implantable lead having an adjustable intermediate region in accordance with embodiments of the present disclosure.

FIG. 4B is a schematic view of another exemplary single pass implantable lead having an adjustable intermediate region in accordance with embodiments of the present disclosure.

FIG. 4C is a schematic view of an exemplary single pass implantable lead having a telescoping intermediate region in accordance with embodiments of the present disclosure.

FIG. 4D is a schematic view of an exemplary single pass implantable lead and intermediate region in accordance with embodiments of the present disclosure.

FIG. 5 is an enlarged schematic view of a region of an exemplary single pass implantable lead in accordance with embodiments of the present disclosure.

FIG. 6 is an exemplary flowchart illustrating a method of implanting a single pass implantable lead adjacent (or around) a left ventricle and left atrium in the coronary venous system of a patient in accordance with embodiments of the present disclosure.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

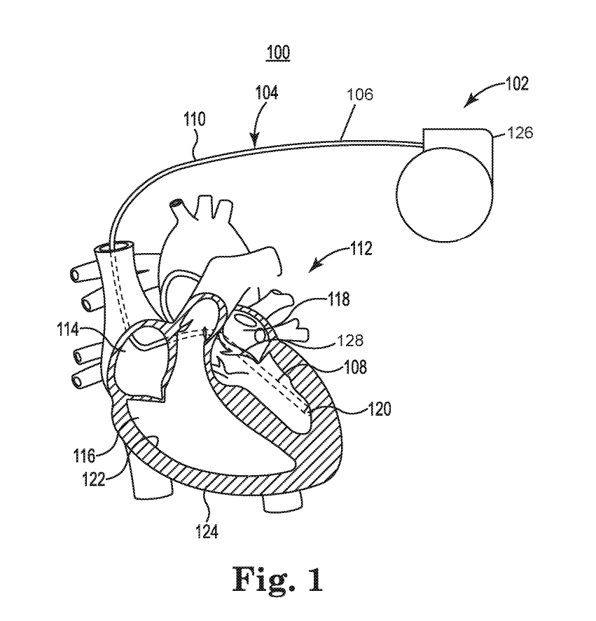

FIG. 1 is a schematic view of an implantable system 100 including an exemplary implantable medical device 102 and an implantable lead 104 in accordance with embodiments of the present disclosure. As shown, the system 100 includes the implantable medical device 102 and the implantable lead 104. As indicated in FIG. 1, the implantable lead 104 includes a proximal end, indicated generally at 106 and a distal end, indicated generally at 108. Further, the implantable lead 104 may include a flexible lead body 110. As shown in FIG. 1, the heart 112 includes a right atrium 114, a right ventricle 116, a left atrium 118, a left ventricle 120, and a mitral valve 128. It can be seen that the heart 112 includes an endocardium 122 covering the myocardium 124.

The implantable medical device 102 may be implanted subcutaneously within an implantation location or pocket in the patient's chest or abdomen. The implantable medical device 102 may be any implantable medical device known in the art or later developed, for detecting a cardiac condition of a patient and/or delivering an electrical therapeutic stimulus to the patient. In various embodiments, the implantable medical device 102 is a bradycardia pacemaker, an implantable cardioverter/defibrillator (ICD), a cardiac resynchronization (CRT) device configured for pacing, and/or includes combinations of pacing, CRT, and defibrillation capabilities.

The implantable medical device 102 may include a header 126. The header 126 may include one or more connector ports (not shown) to couple the implantable lead 104 to the implantable medical device 102. The one or more connector ports of the header electrically and physically contacts a connector assembly (not shown) of the implantable lead 104. The header 126 is attached to a hermetically sealed enclosure that contains a battery, electronic circuitry, and other components known to those skilled in the art. Electrical contacts (not shown) in the header 126 are any type known to those skilled in the art that are electrically connected via feedthroughs (not shown) mounted to extend through the hermetically sealed enclosure in order to electrically couple the implantable lead 104 with implantable medical device 102. Exemplary connectors that may be used in conjunction with the implantable medical device 102 can include, but are not limited to, a quadripolar (e.g., IS4 or similar) connector, a bipolar (e.g., IS1 or similar) connector, or an IS1 plus IS4, or an IS4 plus IS4 connected by a Y adaptor. The electronic circuitry, included with the header 126 and while not shown, is configured to a detection/energy delivery system configured to receive cardiac rhythm signals from the electrode(s) (not shown) provided with the implantable lead 104.

The lead body 110 of the lead 104 can be made from any flexible, biocompatible material suitable for lead construction. In various embodiments, the lead body 110 is made from a flexible, electrically insulative material. In one embodiment, the lead body 110 is made from silicone. In another embodiment, the lead body 110 is made from polyurethane. In various embodiments, respective segments of the lead body 110 are made from different materials, so as to tailor the lead body 110 characteristics to its intended clinical and operating environments. In various embodiments, proximal and distal ends of the lead body 110 are made from different materials selected to provide desired functionalities.

The implantable lead 104 may be a bipolar pacing lead including a single terminal pin and ring electrode. In addition, the implantable lead 104 may be a pacing lead (e.g., bradycardia) or a CRT lead with two or more low-voltage electrodes. The implantable lead 104 may include one or more left ventricle (LV) electrodes and one or more left atrium (LA) electrodes. The lead 104 may include electrodes for sensing the electrical activity of the heart 112 and/or applying a stimulating pulse to left atrium 118 and/or the left ventricle 120. The implantable lead 104 may be a VDD single pass lead. The implantable lead 104 may include sensing capabilities (e.g., a pressure sensing/pacing lead with a quadripolar type connector). More specifically, during therapy delivery, the circuitry provided with the header 126 controls electrodes provided with the implantable lead 104 to detect and/or measuring various physiological parameters. Example parameters that can be detected and/or measured include, but are not limited to, transthoracic impedance, respiratory rate, minute ventilation, heart rate, heart rate variability, cardiac dysynchrony, activity, posture, blood chemistry, O2 saturation, heart sounds, wall stress, strain, hypertrophy, inter-electrode impedance, electrical timing delays (e.g., AV interval, Q-LV interval, etc.), cardiac pressure (e.g., RA and/or coronary venous pressure), cardiac output, temperature, depolarization amplitudes, and depolarization timing. Information from one or more of these physiological parameters may be used to adjust operating parameters such as the amplitude, timing, and/or pulse width of the stimulus energy delivered to the lead 104 from the implantable medical device 102. Other types of leads and/or lead connector types can also be used in conjunction with the implantable medical device 102, and other lead configurations are also possible, as desired.

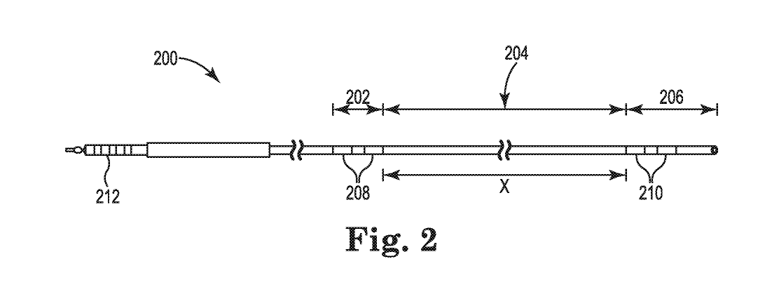

FIG. 2 is a schematic view of an exemplary single pass implantable lead 200 in accordance with embodiments of the present disclosure. In certain instances and as discussed in further detail below, the single pass implantable lead 200 may provide pacing to the left atrium (LA) and the left ventricle (LV) of a patient, and also obviate the need for a pacing lead in the right atrium (RA) and the right ventricle (RV). The single pass implantable lead 200 includes a proximal region 202, a distal region 206, and an intermediate region 204 between the proximal region 202 and the distal region 206. The proximal region 202 includes one or more proximal electrodes 208, and the distal region 206 includes one or more distal electrodes 210. The one or more proximal electrodes 208 and/or the one or more distal electrodes 210 may be configured to sense and/or pace a patient's heart. The proximal region 202 may include one, two, three, or four proximal electrodes 208. Similarly, the distal region 206 may include one, two, three, or four distal electrodes 210. In addition, the proximal region 202 may have a greater thickness (not shown) than the thickness of the distal region 206. In certain instances, the proximal region 202 may be thicker to enhance contact of the one or more proximal electrodes 208 against a vessel wall. As is shown in FIG. 2, the proximal region 202 includes two proximal electrodes 208 and the distal region 206 includes two distal electrodes 210. In certain instances, multiple proximal electrodes 208 may be coupled to a common conductor. As a result, the multiple proximal electrodes 208 may be configured to sense or pace simultaneously or in a coordinated manner. In addition, multiple distal electrodes 210 may be coupled to a common conductor, and thus may be configured to sense or pace simultaneously or in a coordinated manner. The proximal electrodes 208 and distal electrodes 210 may be connected to different conductors.

Due to the spacing between the proximal region 202 and the distal region 206, the proximal electrodes 208 and the distal electrodes 210 may sense and/or pace different locations of the heart. For example, the distance “X” provided by the intermediate region 204, separating the proximal electrodes 208 and the distal electrodes 210, may be provided such that the proximal electrodes 208 and the distal electrodes 210 are positionable, respectively, on the epicardial surface of the left atrium and the left ventricle of the heart via the coronary veins. The distance “X” may be between 4.0 cm and 150 cm. As a result, the proximal electrodes 208 may be configured to sense and pace the left atrium of the patient, and the distal electrodes 210 may be configured to sense and pace the left ventricle of the patient. Although not shown, the single pass implantable lead 200 may be coupled to an implantable medical device via a connector 212.

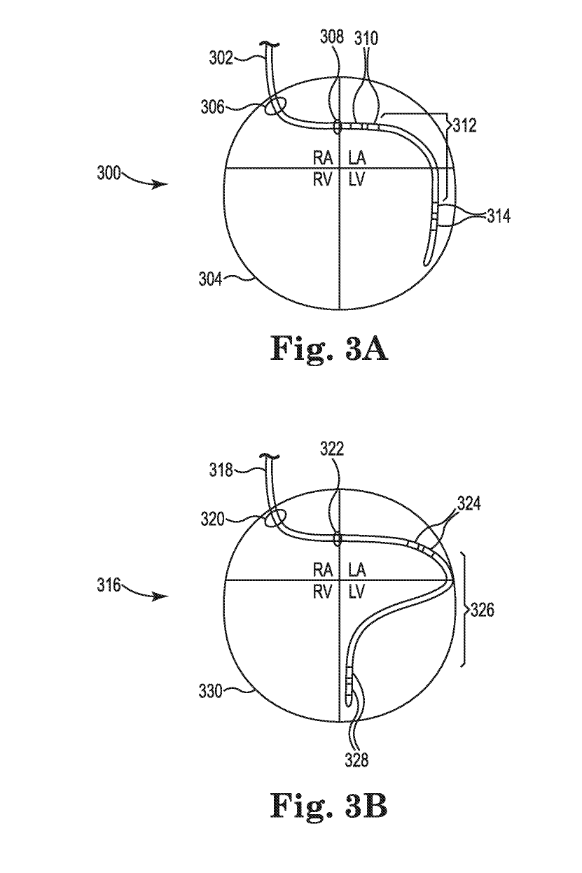

FIG. 3A is an illustration 300 of an exemplary placement of a single pass implantable lead 302 in a patient's heart 304 in accordance with embodiments of the present disclosure. The illustration 300 shows the heart 304 broken into the four quadrants: right atrium (RA), right ventricle (RV), left atrium (LA), and left ventricle (LV). The single pass implantable lead 302 is shown entering the heart 304 via the superior vena cava 306, and routed epicardially around the LA through the coronary sinus ostium (CS OS) 308. A region of the single pass implantable lead 302 around the LA includes one or more proximal electrodes 310. The one or more proximal electrodes 310 may be anchored in and/or contact the great cardiac vein (GCV) or coronary sinus (CS) in a way to be adjacent to the myocardium of the LA. From this position, the single pass implantable lead 302 is further routed through the vasculature of the heart 304 to a position around the LV. The region of the single pass implantable lead 302 that is routed between the LA electrodes (310) and the LV electrodes (314) is an intermediate portion 312 that may not include any electrodes. A region of the single pass implantable lead 302 on the LV includes one or more distal electrodes 314. The one or more distal electrodes 314 may be positioned, for example in the coronary branch of the heart 304.

FIG. 3B is an illustration 316 of another exemplary placement of a single pass implantable lead in a patient's heart 330 in accordance with embodiments of the present disclosure. Similar to the illustration 300 shown in FIG. 3A, the illustration 316 shows the heart 330 broken into the four quadrants: right atrium (RA), right ventricle (RV), left atrium (LA), and left ventricle (LV). The single pass implantable lead 318 is again shown entering the heart 330 via the superior vena cava 320, and routed epicardially around the LA through the coronary sinus ostium (CS OS) 322. A region of the single pass implantable lead 318 on the LA includes one or more proximal electrodes 324, which may be anchored in the great cardiac vein (GCV) or coronary sinus (CS) in a way to be adjacent to the myocardium of the LA. From this position, the single pass implantable lead 318 may be further routed through the vasculature of the heart 330 to a position on or adjacent the LV. In the schematic shown in FIG. 3B, the single pass implantable lead 318 is routed further within the vasculature toward the septum between the LV and right ventricle (RV). An intermediate portion 326 that may not include any electrodes provides a sufficient length to allow for this routing. In addition, a region of the single pass implantable lead 318 on or adjacent the LV includes one or more distal electrodes 328. The one or more distal electrodes 328 may be positioned, for example in the anterior vein adjacent the ventricular septum of the heart 330.

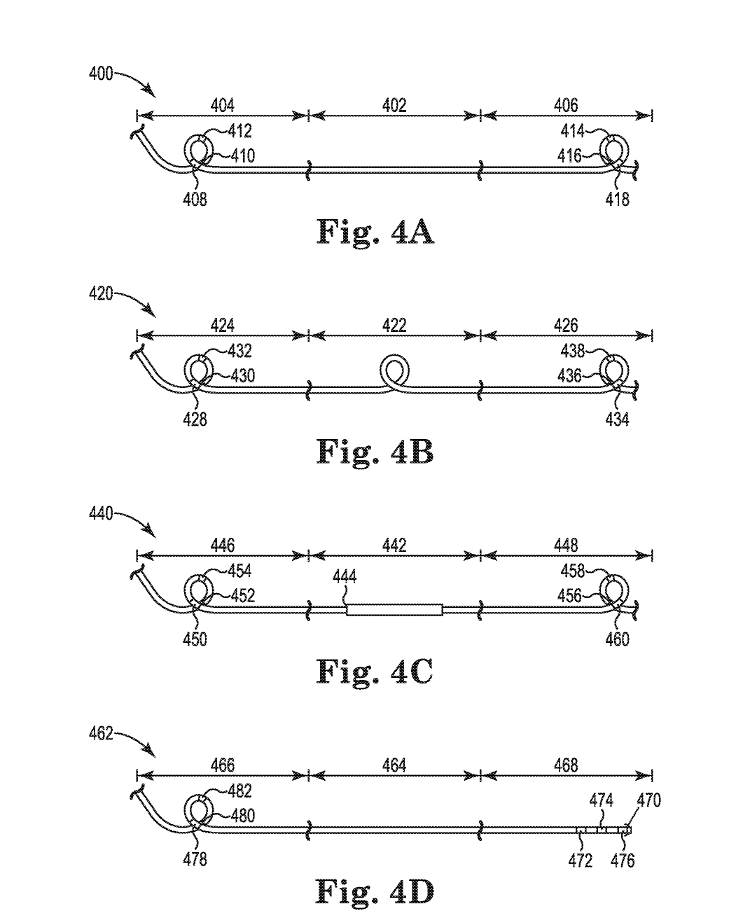

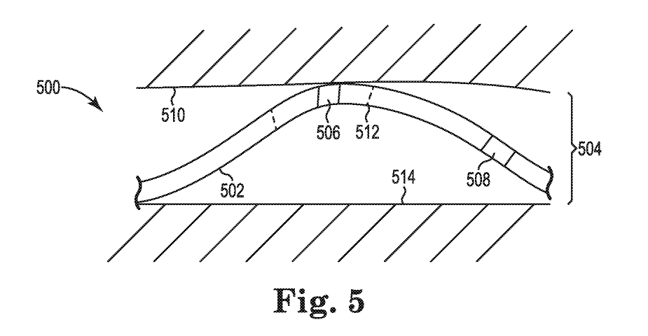

FIG. 4A is a schematic view of an exemplary single pass implantable lead 400 having an adjustable intermediate region 402 in accordance with embodiments of the present disclosure. The intermediate region 402 may comprise a flexible region that allows for damping of forces that may pull or push the single pass implantable lead 400 when implanted. As described in further detail below with reference to FIG. 5, a proximal region 404 and/or a distal region 406 of the single pass implantable lead 400 may include anchoring features that may be provided by biasing the proximal region 404 and the distal region 406. In addition and unlike the intermediate region 402, the proximal region 404 and the distal region 406 respectively include proximal electrodes 408, 410, 412 and distal electrodes 414, 416, 418. In certain instances, the intermediate region 402 does not include or is free of electrodes. Although the single pass implantable lead 400 is shown as including three proximal electrodes 408, 410, 412 and three distal electrodes 414, 416, 418, other electrode quantities are also contemplated. The proximal region 404 and the distal region 406 may be biased, as shown in FIG. 4A, to contact a vessel wall and provide anchoring of the proximal region 404 and the distal region 406 thereto. Anchoring in this manner may be atraumatic to the vessel wall. As a result of the adjustable intermediate region 402 comprising a flexible material that allows for the adjustable intermediate region 402 to compress and stretch (and/or expand and contract), forces that may alter or displace the anchoring function of the proximal region 404 and the distal region 406 are dampened or absorbed by the adjustable intermediate region 402, which enhances anchoring and accurate placement of the single pass implantable lead 400. In addition, the adjustable intermediate region 402 may be adjustable by including a material composition having a lower durometer than the proximal region 404 and the distal region 406. Alternatively or in addition thereto, the adjustable intermediate region 402 may be adjustable by including a material composition having a lesser thickness than the proximal region 404 and the distal region 406. As a result, the adjustable intermediate region 402 may have a greater flexibility and/or elasticity than the proximal region 404 and the distal region 406.

FIG. 4B is a schematic view of another exemplary single pass implantable lead 420 having an adjustable intermediate region 422 in accordance with embodiments of the present disclosure. As shown in FIG. 4B, the intermediate region 422 may comprise slack, similar to an accordion or coil, that allows for damping of forces that may pull or push the single pass implantable lead 420 when implanted. A proximal region 424 and a distal region 426 of the single pass implantable lead 420 may include biased portions having, respectively, proximal electrodes 428, 430, 432 and distal electrodes 434, 436, 438. The slack in the intermediate region 422 allows for the proximal electrodes 428, 430, 432 and the distal electrodes 434, 436, 438 to remain in contact with a vessel wall by way of the intermediate region 422 absorbing or mitigation forces that may displace the anchoring provided by the biased portions of the proximal region 424 and the distal region 426. The biased portions of the proximal region 424 and the distal region 426 may provide anchoring of the single pass implantable lead 420 without having to puncture, penetrate, or cause damage to the vessel wall of the heart, i.e., anchoring may be achieved in an atraumatic manner.

FIG. 4C is a schematic view of an exemplary single pass implantable lead 440 having a telescoping intermediate region 442 in accordance with embodiments of the present disclosure. As shown in FIG. 4C, the telescoping intermediate region 442 includes at least one overlapping region 444 of material that allow for the telescoping intermediate region 442 to stretch and contract. In this manner, the telescoping intermediate region 442 allows for damping of forces that push and pull on the single pass implantable lead 440. Portions on the single pass implantable lead 440 on either side of the telescoping intermediate region 442 may be configured to translate in and out of the telescoping intermediate region 442. In certain instances, only a distal region of the single pass implantable lead 440 may telescope in and out of the telescoping intermediate region 442. In other instances, only a proximal region of the single pass implantable lead 440 may telescope in and out of the telescoping intermediate region 442. As a result, the telescoping intermediate region 442 allows for stable placement of a proximal region 446 and a distal region 448 of the single pass implantable lead 440. The proximal region 446 and the distal region 448 may include biased portions having, respectively, proximal electrodes 450, 452, 454 and distal electrodes 456, 458, 460. The biased portions may ensure that the proximal electrodes 450, 452, 454 and the distal electrodes 456, 458, 460 contact, and are anchored to, a vessel wall of the heart in a way that at least one electrode contacts the myocardium wall side of the LA and LV. Anchoring in this manner may be atraumatic to the vessel wall. The telescoping intermediate region 442 a length adjustment of the single pass implantable lead 440, which enhances the ability of the anchoring provided by the biased portions of the proximal region 446 and the distal region 448. Although not explicitly shown, the thickness of the proximal region 446 and the distal region 448 may be equal. In other instances, the thickness of the proximal region 446 may be greater than the thickness of the distal region 448, and vice versa.

FIG. 4D is a schematic view of an exemplary single pass implantable lead 462 and intermediate region 464 in accordance with embodiments of the present disclosure. As shown in FIG. 4D, the intermediate region 464, as opposed to a proximal region 466 and a distal region 468 of the single pass implantable lead 462, does not include any electrodes. The proximal region 466 and the distal region 468 include proximal electrodes 478, 480, 482 and distal electrodes 472, 474, 476. The distal region 468 is shown as including a tine 470 configured to anchor the distal region 468 to a vessel wall. The proximal region 466 is shown as having a biased portion which is configured to anchor the proximal region 466 to the vessel wall. Although not explicitly shown, the proximal region 466 may also include a tine configured to anchor the proximal region 466 to the vessel wall. Further, one or potion of the proximal region 466 and the distal region 468 may include a bias to enhance contact of the proximal electrodes 478, 480, 482 and distal electrodes 472, 474, 476 against the vessel wall in a way that at least one electrode contacts the myocardial wall side of the vessel 510 accordingly. Anchoring in this manner may be atraumatic to the vessel wall

In each of the arrangements shown in FIGS. 4A-4D, the illustrative single pass implantable lead is not intended to suggest any limitation as to the scope of use or functionality of embodiments of the present invention. Neither should the illustrative single pass implantable lead be interpreted as having any dependency or requirement related to any single component or combination of components illustrated therein. Additionally, any one or more of the components depicted in FIG. 4 may be, in embodiments, integrated with various ones of the other components depicted therein (and/or components not illustrated), all of which are considered to be within the ambit of the present invention. For example, the single pass implantable lead 400, shown in FIG. 4A, may include a tine, as shown in FIG. 4D, or a telescoping intermediate region as is shown in FIG. 4C.

FIG. 5 is an enlarged schematic view of a region 500 of an exemplary single pass implantable lead 502 in accordance with embodiments of the present disclosure. As shown in FIG. 5, the implantable lead 502 is inserted at a target region within a blood vessel 504. The enlarged region 500 of the implantable lead 502 shown may be either a proximal region or a distal region as discussed above with reference to FIGS. 2, and 4A-D.

Each region 500 of the implantable lead 502 may include one or more electrodes. As shown in FIG. 5, the region 500 includes two electrodes 506, 508, each of which may be coupled to a corresponding cable conductor or coil conductor within the interior of the lead 502. An implantable medical device such as a pulse generator may supply electrical pulses to the electrodes 506, 508 for pacing the heart and/or for sensing cardiac electrical activity. Similarly, the implantable medical device may supply electrical pulses to electrodes in the other region of the implantable lead 502 (not shown) for pacing the heart and/or for sensing cardiac electrical activity.

Each region 500 of the implantable lead 502 may be configured to anchor within the vessel 504. Each region 500 of the implantable lead 502 may be configured to anchor by pre-biasing of one or more portions of the implantable lead 502. Pre-biasing of one or more portions (either one of or both of the proximal region and the distal region) may configure the implantable lead 502 to engage a wall of blood vessel to secure the implantable lead 502 in a location along the vessel. Engagement of the blood vessel wall may be such that the vessel wall is not penetrated by the lead 502. The biasing may occur by way of a material transition region 512 that provides a natural turn or curve in the implantable lead 502 due to the transition. The material transition region 512 may be provided proximal or distal to at least one of the electrodes 506, 508. The material transition region may be the result of the implantable lead 502 transitioning between polyurethane and silicone. The anchoring allows for positioning of the implantable lead 502 within the vessel 504. As shown in FIG. 5, the implantable lead 502 is anchored against the myocardial wall side 510 of the vessel rather than the myocardium-free wall side 514 of the vessel 504. As a result, the implantable lead 502 may be secured in place such that the electrodes 506, 508 contact the myocardial wall side 510 of the vessel 504 to provide pacing to the desired cardiac chamber (e.g., LA or LV) and/or for sensing cardiac electrical activity.

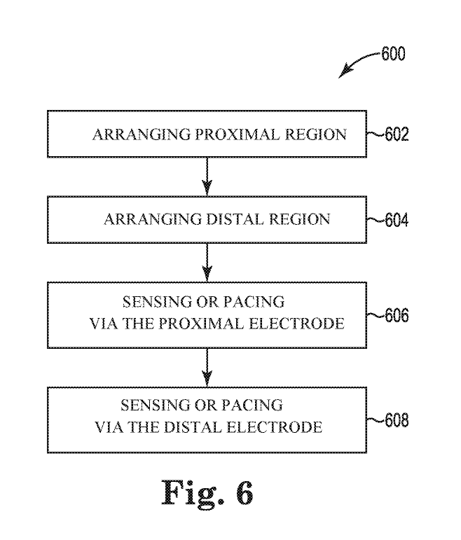

FIG. 6 is an exemplary flowchart 600 illustrating a method of implanting a single pass implantable lead in a left ventricle and left atrium of a patient in accordance with embodiments of the present disclosure. As is shown at block 602, the method comprises arranging a proximal region of the single pass implantable lead including at least one proximal electrode adjacent or near the left atrium of the patient. This step may include arranging the proximal region against the left atrial epicardial wall of the patient. In addition, as is shown at block 604, the method also comprises arranging a distal region of the single pass implantable lead including at least one distal electrode in the left ventricle of the patient. This may include arranging the distal section against the left ventricular epicardial wall of the patient.

In this method, the single pass implantable lead may provide pacing to the left atrium (LA) and the left ventricle (LV) of a patient, and also obviate the need for a pacing lead in the right atrium (RA) and the right ventricle (RV). Using a single pass lead reduces the time of implant, eases extraction (and/or possible repositioning) of the single lead, and also reduces the number of leads provided in the heart chamber. Further, arranging the single pass implantable lead in the left atrium and left ventricle of the patient, as described, avoids crossing the tricuspid valve of the patient, and may avoid crossing any valves. For example, the arranging the proximal region may include arranging the lead (and proximal electrode(s)) in the patient's great cardiac vein (GCV) or coronary sinus. From this position, the distal region (and distal electrode(s)) may be further arranged in the patient's coronary branch.

Further, as is shown at block 606, the method comprises sensing or pacing a cardiac rhythm of the left atrium of the patient via the at least one proximal electrode. As is shown at block 608, the method comprises sensing or pacing the left ventricle of the patient via the at least one distal electrode. In certain instances, sensing or pacing via the at least one distal electrode may provide a strong enough electrical impulse such that the right ventricle of the patient is also stimulated (e.g. distal electrode placed in the anterior vein). The method also may include coupling the single pass implantable lead to an implantable medical device. Alternatively or in addition thereto, the method can also include sensing or pacing the left atrium of the patient via the at least one proximal electrode. Further, the method may also comprise sensing or pacing cardiac rhythm of the left ventricle of the patient via the at least one distal electrode. The implantable medical device may be a pacemaker or a CRT device. The single pass implantable lead may be configured to deliver pacing therapy or CRT therapy to the left atrium and the left vertical of the patient.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. An apparatus comprising:
an implantable medical device; and
a single pass implantable lead configured to be coupled to the implantable medical device and arranged near or in a left ventricle and left atrium of a patient, the implantable medical lead comprising:
a flexible lead body having a proximal region configured to anchor the flexible lead body against an epicardial surface of a left atrial epicardial wall, a distal region configured to anchor the flexible lead body to a left ventricular epicardial wall, and an adjustable intermediate region between the proximal region and distal region;
at least one proximal electrode arranged in the proximal region of the flexible lead body and configured to sense and pace the left atrium of the patient; and
at least one distal electrode arranged in the distal region of the flexible lead body and configured to sense and pace the left ventricle of the patient.

2. The apparatus of claim 1, wherein the implantable medical device including a header having a connector port, and the single pass implantable lead is coupled to the implantable medical device via the connector port.

3. The apparatus of claim 2, wherein the implantable medical device is at least one of a Cardiac Resynchronization Therapy (CRT) device and a bradycardia pacemaker.

4. The apparatus of claim 1, wherein the at least one proximal electrode is configured to sense and pace the left atrium of the patient in the great cardiac vein (GCV) or coronary sinus, and wherein at least one distal electrode is configured to sense and pace the left ventricle of the patient in the patient's coronary branch.

5. The apparatus of claim 1, wherein the adjustable intermediate region of the flexible lead body comprises an adjustable length, the adjustable length being configured to compress and stretch.

6. The apparatus of claim 1, wherein the adjustable intermediate region comprises a first thickness, and the proximal and distal regions comprise a second thickness, and the first thickness differs from the second thickness.

7. The apparatus of claim 1, wherein the intermediate length is between 40 mm and 150 mm.

8. The apparatus of claim 1, wherein at least one of the proximal region, the distal region, and the intermediate region comprises polyurethane, and wherein at least one of the proximal region, the distal region, and the intermediate region comprises silicone.

9. The apparatus of claim 8, further comprising a material transition portion between the polyurethane and silicone, and wherein the material transition region is configured to provide a bias.

10. The apparatus of claim 9, wherein the material transition region is proximal to the at least one proximal electrode or the at least one distal electrode.

11. The apparatus of claim 10, wherein the material transition region is proximal to the at least one proximal electrode, and the at least one proximal electrode contacts the left atrium of the patient in the great cardiac vein (GCV) or coronary sinus.

12. A single pass implantable lead comprising:
a flexible lead body having a proximal region configured to anchor the flexible lead body against an epicardial surface of a left atrial epicardial wall of a patient, a distal region configured to anchor the flexible lead body to a left ventricular epicardial wall of the patient, and an adjustable intermediate region between the proximal region and distal region, the adjustable intermediate region being configured to configured to mitigate displacement forces to enhance anchoring of the proximal region or distal region;
at least one proximal electrode arranged in the proximal region of the flexible lead body and configured to sense and pace the left atrium of the patient; and
at least one distal electrode arranged in the distal region of the flexible lead body and configured to sense and pace the left ventricle of the patient.

13. The single pass implantable lead of claim 12, wherein the adjustable intermediate region of the flexible lead body comprises an adjustable length.

14. The single pass implantable lead of claim 12, wherein the adjustable intermediate region is configured to compress and stretch to mitigate displacement forces to enhance anchoring of the proximal region or distal region.

15. The single pass implantable lead of claim 12, wherein at least one of the proximal region and the distal region having a pre-biased shape configured to engage a wall of a blood vessel to secure the lead.

16. The single pass implantable lead of claim 12, wherein the proximal region and the distal region comprise a pre-biased shape configured to engage a wall of a blood vessel to secure the lead.

17. A method of implanting a single pass implantable lead in coronary venous system adjacent to a left ventricle and left atrium of a patient, the method comprising:
arranging a proximal region the single pass implantable lead including at least one proximal electrode against the left atrium epicardial wall of the patient;
arranging a distal region the single pass implantable lead including at least one distal electrode against the left ventricle epicardial wall of the patient, the single pass implantable lead having an adjustable intermediate region between the proximal region and the distal region;
sensing or pacing a cardiac rhythm of the left atrium of the patient via the at least one proximal electrode; and
sensing or pacing the left ventricle of the patient via the at least one distal electrode.

18. The method of claim 17, wherein arranging the proximal region comprises arranging the at least one proximal electrode in the patient's great cardiac vein (GCV) or coronary sinus.

19. The method of claim 18, wherein arranging the distal region comprises arranging at least one distal electrode in the patient's coronary branch.

20. The method of claim 17, further comprising coupling the single pass implantable lead to an implantable medical device, wherein the implantable medical device is at least one of a Cardiac Resynchronization Therapy (CRT) device and a bradycardia pacemaker.
